(19) United States
(12) Patent Application Publication
(10) Pub. No.: US 2008/0051826 A1
(43) Pub. Date: Feb. 28, 2008

(54) CUPPING JAR WITH LAMP

(76) Inventor: Yun-Sang Cho, Gwangju Metropolitan City (KR)

Correspondence Address: MERCHANT & GOULD PC, P.O. BOX 2903, MINNEAPOLIS, MN, 55402-0903, US

(21) Appl. No.: 11/845,568

(22) Filed: Aug. 27, 2007

(30) Foreign Application Priority Data
Aug. 28, 2006 (KR) 10-2006-0088507
Sep. 1, 2006 (KR) 10-2006-0088508

(51) Int. Cl. A61B 17/00; A61B 19/00; B65D 25/28

(52) U.S. Cl. 606/201; 206/438; 220/756

(57) ABSTRACT

A fire cupping jar includes a cup portion and a lamp that is provided in the cupping jar. The cupping jar can be repeatedly used without needing to be replenished with alcohol and can use fire capable of providing uniform flame.

### CROSS-REFERENCE TO RELATED APPLICATION

This application claims under 35 U.S.C. §119(a) on Korean Patent Application No. 10-2006-0088507 filed on Aug. 28, 2006 and Korean Patent Application No. 10-2006-0088508 filed on Sep. 1, 2006, the entire contents of which are incorporated herein by reference.

### BACKGROUND OF THE INVENTION

(a) Field of the Invention

The present invention relates to a cupping jar that can be repeatedly used without needing to be replenished with alcohol and can use fire capable of providing uniform flame.

(b) Background Art

Cupping therapy is a medical treatment that forms negative pressure inside a cupping jar using heat or a negative pressure device, and places the cupping jar on the skin so as to draw blood, to cause local congestion, or to apply a physical stimulus, thereby treating a disease. The kinds of cups used for cupping are small cups, bowls, or drums. Cupping therapy is widely used for treating traumatic diseases such as a simple bruise, as well as chronic internal diseases.

Cupping therapy has been used since ancient times and was a popular practice in both Western and Eastern medicine. In Eastern medicine, fire cupping therapy has been used over a long period of time in traditional Chinese treatment. In Western medicine, cupping therapy has been used since the Roman and Greek eras.

The effects of cupping therapy are as follows. Metabolism and blood purification are speeded up due to gas exchange, blood circulation and hematogeneous function are improved, and wastes and poisonous matters are excreted. In addition, the acid and base in the body fluids are balanced, and immunity is increased.

In the past, a fire cupping jar, which has been used for cupping treatment, heats air by fire to form a vacuum. Specifically, cotton was ignited and put in a pottery cup, a horn, or a bamboo cup, and then the cupping jar was placed on the skin. Then, oxygen and hydrogen in the cupping jar were burned up, such that the density of air in the cupping jar was lowered, causing the cupping jar to be anchored to the skin, and warming, gas exchange, and negative pressure and the like occurred.

Recently, in order to prevent alcohol from running down the cupping jar during cupping treatment, diatomite having good alcohol absorptiveness or a porous material is provided in the cupping glass, or ignition cotton is fixed within the cupping jar by a steel wire.

However, in such a cupping jar, heating power varies according to the amount of alcohol in the cupping jar, and alcohol needs to be repeatedly replenished.

### SUMMARY OF THE INVENTION

The present invention has been made in an effort to provide a fire cupping jar that can maintain a flame for a long time regardless of the absorption amount of alcohol.

According to an aspect of the invention, a fire cupping jar includes a lamp 6, 6a, such as an alcohol lamp, within the cupping jar. The lamp 6, 6a includes a storage unit that is filled with an absorbent for absorbing a volatile combustible material, such as alcohol, and an ignition unit that ignites the lamp. Preferably, the ignition unit includes a combustion core, where alcohol combustion occurs, and alcohol is continuously and regularly supplied to the combustion core, thereby maintaining uniform flame. Therefore, the fire cupping jar can be repeatedly used without being replenished with alcohol, and the intensity of a flame can be uniformly maintained. Since a uniform flame is maintained in the cupping jar, the temperature within the cupping jar is maintained constant, and oxygen and hydrogen are burned up and removed from the air in the cupping jar, thereby improving a negative pressure effect. The cupping jar may include a plurality of lamps and/or a plurality of combustion cores.

In the cupping jar, the absorbent used to absorb a large amount of combustible material in the lamp 6, 6a, such as an alcohol lamp, may be an alcohol-soaked cotton 9.

During the cupping treatment, the temperature of the fire cupping jar generally increases, and as a result, an operator may burn his/her hands when taking the cupping jar. In order to solve this problem, a handle portion 2 may be provided in an upper portion of the cupping jar. The handle portion 2 may be formed of a material different from the cup portion 1 and attached thereto by a screw 5 and a screw fixing insert or an adhesive. The upper portion of the cupping jar itself may form the handle portion 2.

When the cupping jar has a handle portion 2, the lamp 6, 6a may be provided inside the handle portion 2. The lamp 6, 6a that is provided inside the handle portion 2 may protrude toward an internal space of the cup portion 1. Also, the lamp may be provided in the internal space of the cup portion 1, not in the handle portion 2 (for example, see FIG. 12). In this case, the lamp may be preferably formed of a metal.

A ventilating opening 4 may be provided at a predetermined position of the cup portion 1, so that an appropriate amount of air is introduced inside the cupping jar. Therefore, the cupping jar can be conveniently attached/detached to/from the skin. An opening/closing means, including a ventilating opening button 20, may be provided to easily open/close the ventilating opening 4.

In the cupping jar, the lamp 6 may be filled with an absorbent, such as cotton. In addition, an alcohol absorbing opening 7 may be provided so as to allow alcohol to be easily absorbed in the cotton.

The cupping jar may further include an ignition device, which enables one to ignite the combustion core without a separate ignition tool. An example of the ignition device may be a device having a function of an electronic lighter.

A material, such as bioceramics, which generates far infrared rays, or a stone material, such as jade, may be attached to the cup.

The cup portion 1 may be formed of a pottery or thermosetting resin. Alternatively, the cup portion 1 may also be formed of glass or reinforced plastic.

The fire cupping jar according to the present invention includes a lamp that is provided within the cupping jar to ignite a fire. In the lamp 6, in which an alcohol-soaked cotton 9 is inserted, the combustion core 3 that is formed of a fibrous material or a porous material having good absorptiveness, such as diatomite, is exposed to the air, such that a flame can be ignited in the combustion core 3.

In order to prevent the cupping jar from slipping due to sweat on the palms when the operator takes the cupping jar, a handle portion 2 is provided. Accordingly, cupping treatment can be easily and conveniently performed. In addition, if necessary, an electronic lighter function is added, which renders a separate ignition tool unnecessary to ignite the combustion core 3.

### BRIEF DESCRIPTION OF THE DRAWINGS

The above and other features and advantages of the present invention will become more apparent by describing in detail preferred embodiments thereof with reference to the attached drawings in which:

FIG. 1 is a cross-sectional view and a perspective view showing a cupping jar according to an embodiment of the invention;

FIG. 2 is an explanatory view of a lamp in the cupping jar;

FIG. 3 is a perspective view of a cupping jar according to an embodiment of the invention;

FIG. 4 is a cross-sectional view showing the internal structure of a cupping jar according to an embodiment of the invention, in which a metal lamp is provided;

FIG. 5 is a cross-sectional view showing the structure of a metal lamp in a cupping jar according to an embodiment of the invention;

FIG. 6 is an explanatory view of a lamp stopper in a cupping jar according to an embodiment of the invention;

FIG. 7 is a cross-sectional view of a cupping jar according to another embodiment of the invention;

FIG. 8 is an explanatory view of an opening/closing unit of a ventilating opening in a cupping jar according to an embodiment of the invention;

FIG. 9 is an explanatory view illustrating a case where an ignition device is attached to a cupping jar according to an embodiment of the invention;

FIG. 10 is a cross-sectional view of a lamp in a cupping jar according to another embodiment of the invention;

FIG. 11 is a cross-sectional view of a cupping jar according to an embodiment of the invention, in which a functional material is provided inside or outside a cup portion according to an embodiment of the invention;

FIG. 12 is a cross-sectional view of a cupping jar according to an embodiment of the invention, in which a metal lamp protrudes; and

FIG. 13 is a cross-sectional view of a cupping jar according to an embodiment of the invention, in which a lamp having a different shape is provided.

### DETAILED DESCRIPTION

Hereinafter, a fire cupping jar according to the embodiments of the invention will be described in detail with reference to the drawings.

The term “fire cupping jar” used herein refers to a cupping jar, in which a vacuum in a cupping jar is caused by fire.

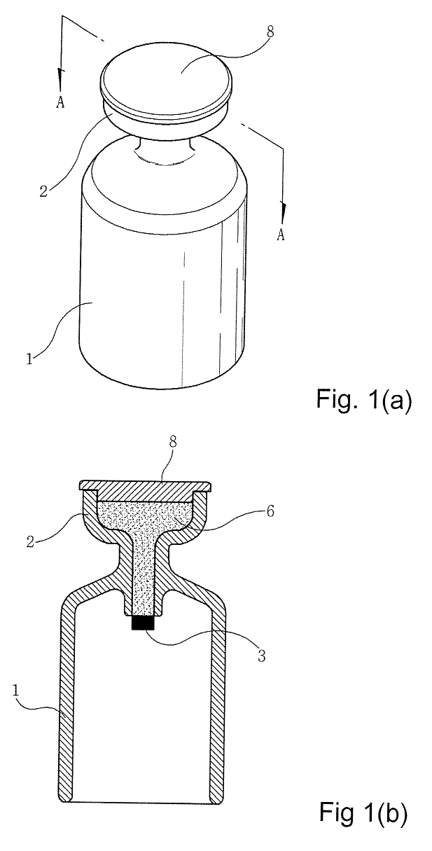

FIG. 1 is a cross-sectional view and a perspective view showing a cupping jar according to an embodiment of the invention. Referring to FIG. 1, a lamp 6 is provided in a handle portion 2. The lamp 6 is filled with an alcohol-soaked cotton 9 or a porous material such that a large amount of alcohol is stably maintained. In order to stably fix a combustion core 3 and the alcohol-soaked cotton 9, an inner wall surface of the lamp may be jagged (not shown). The combustion core 3 is exposed outside the lamp 6 by a predetermined length, such that a uniform flame is maintained in the cup portion 1.

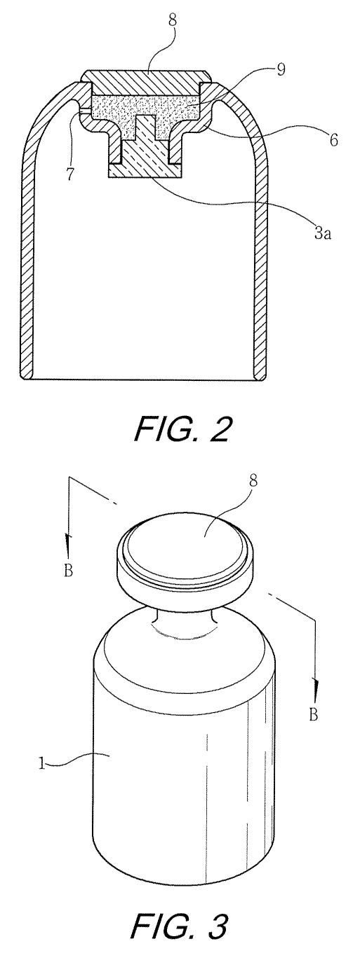

FIG. 2 is an explanatory view of a lamp in a cupping jar according to an embodiment of the invention. Referring to FIG. 2, a lamp 6 is provided in the cup portion 1, not in the handle portion 2. Further, a combustion core 3a that is formed of a porous hard material is connected to an alcohol-soaked cotton 9 for absorbing combustible material, such as alcohol, such that alcohol in the lamp 6 can be stably burned.

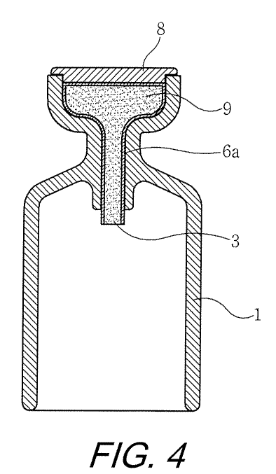

FIGS. 3 and 4 show an example of a cupping jar that includes a metal lamp having an alcohol storage unit. Alcohol is stored in a handle portion 2, and a separate metal lamp 6a is fixedly provided. An end of the handle portion 2 is covered with an openable stopper 8 having a good sealing property. The stopper 8 can be opened, as occasion demands, for example, when the alcohol-soaked cotton 9 or alcohol needs to be replenished.

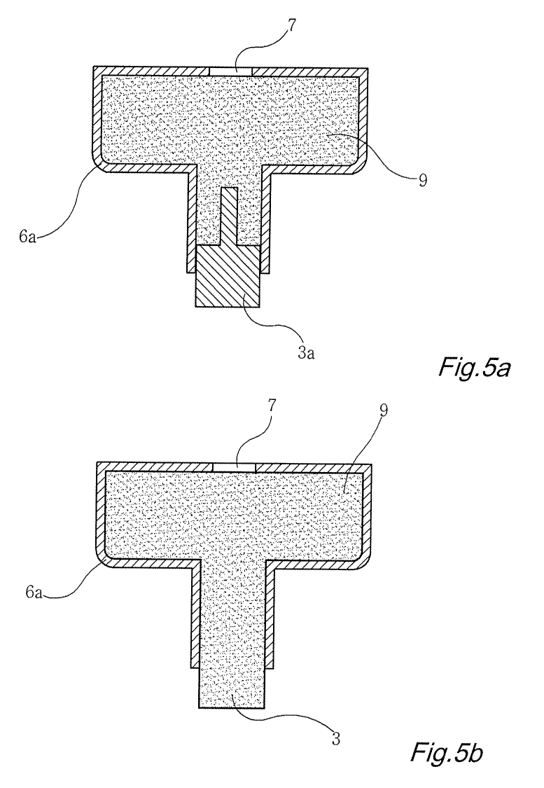

FIG. 5 is a cross-sectional view showing the structure of the metal lamp. The metal lamp 6a that is fixedly provided in the cupping jar is filled with the alcohol-soaked cotton 9 for absorbing the combustible material, such as alcohol. The combustion core 3 that is exposed to the air is formed of a fibrous material, such as cotton. A combustion core 3a formed of a porous hard material may be provided. In addition, an alcohol absorbing opening 7 may be provided in a body portion of the metal lamp as occasion demands.

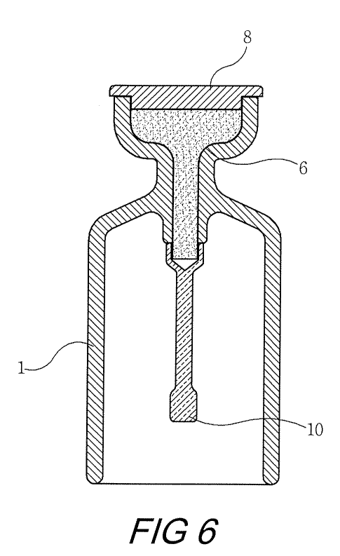

FIG. 6 is an explanatory view of a lamp stopper 10 and illustrates the structure of the stopper 10 which can cover the combustion core 3. When the cupping jar is not used over a long period of time, the lamp stopper 10 prevents alcohol in the lamp 6 from being evaporated, such that a cupping treatment can be repeatedly performed without needing to be replenished with alcohol.

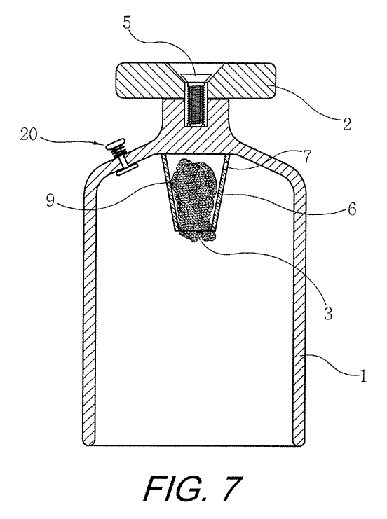

FIG. 7 is a cross-sectional view of a cupping jar according to another embodiment of the invention. Referring to FIG. 7, a handle portion 2 that is formed of a material different from a cup portion 1 is connected to the cup portion 1 by a screw, and a ventilating opening 4 is provided to introduce air inside the cup portion 1.

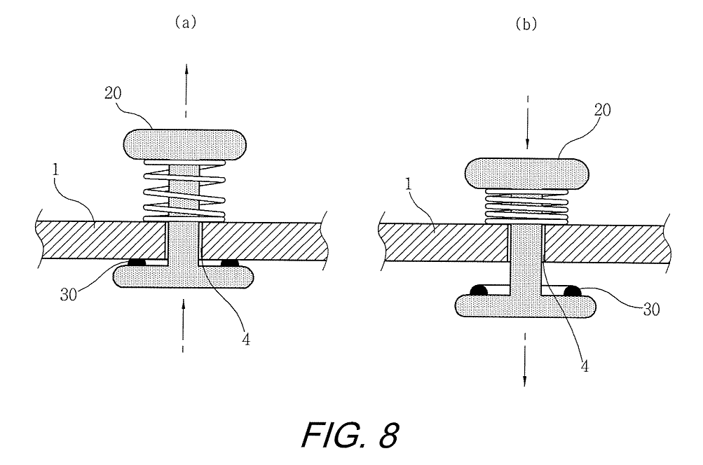

FIG. 8 is an explanatory view of an opening/closing unit of the ventilating opening 4 in the cupping jar according to another embodiment of the invention. The ventilating opening 4 is formed to have a predetermined diameter in a portion of the cup portion 1 to enable air flow into the cup portion 1, if necessary. The ventilating opening 4 is closed by a simple switch. As shown in FIG. 8, if an opening/closing button 20 of the ventilating opening 4 pressed, a closing ring 30 is opened to enable the air flow in.

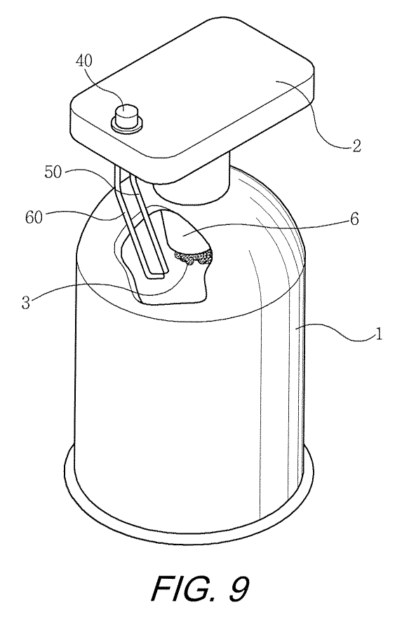

FIG. 9 is an explanatory view illustrating a case where an ignition device is attached to a cupping jar according to another embodiment of the invention. A general electronic lighter is provided, such that the operator can easily ignite the combustion core 3 in the cupping jar. An ignition plug line 50 and a gas discharge pipe 60 extend into the cupping glass 1. Accordingly, when the operator pushes an ignition button, gas is ejected from the end of the gas ejection pipe 60 and a flame is generated at the end of the ignition plug line 50, such that the combustion core 3 is ignited. In addition, portions where the gas ejection pipe 60 and the ignition plug line 50 pass through the wall of cupping jar need to be sealed airtight so as not to cause an air flow.

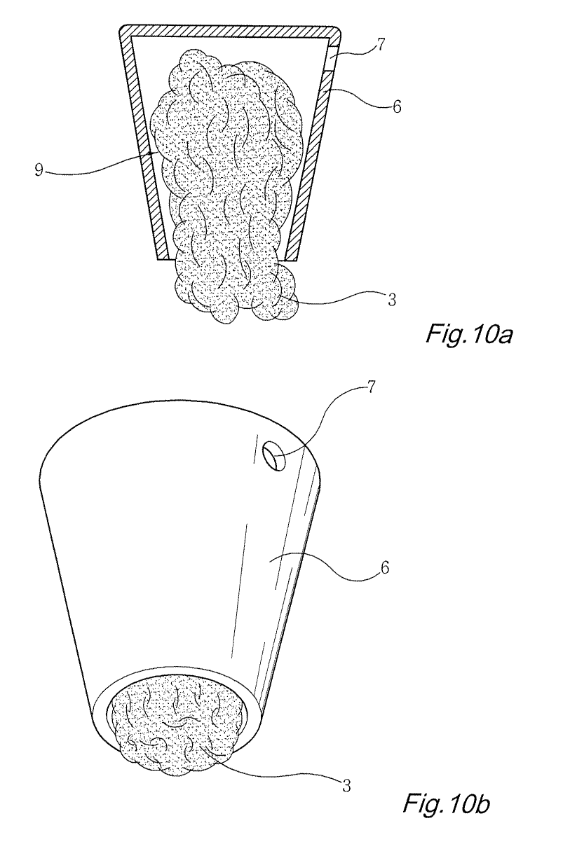

FIG. 10 is a cross-sectional view of a lamp in the cupping jar according to another embodiment of the invention. A space is formed in the lamp 6 to fixedly store a predetermined amount of alcohol-soaked cotton 9. In this case, part of the alcohol-soaked cotton 9 may be exposed outside the lamp 6, such that the exposed part can function as the combustion core 3.

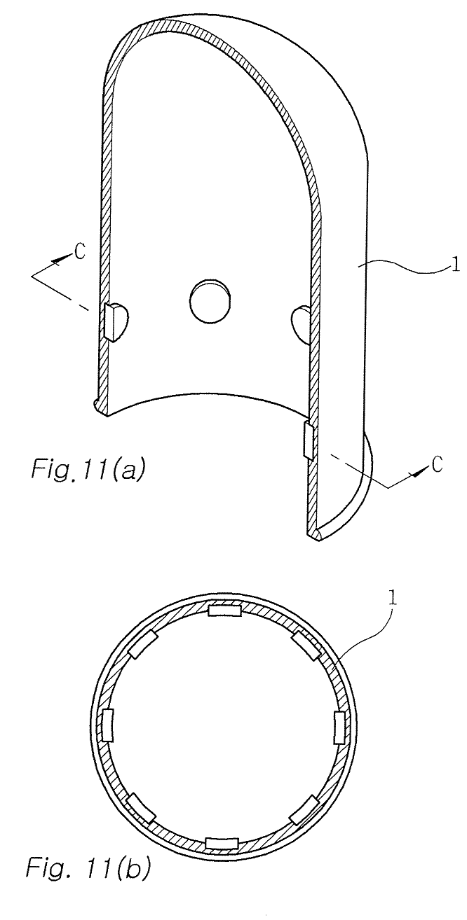

FIG. 11 shows a case where a material, such as bioceramics, which generates far infrared rays, or a functional material, such as a gade, is fixed inside or outside the cup portion 1 so as to increase the effect of the cupping treatment, thereby increasing an effect to maintain the temperature of the cupping glass.

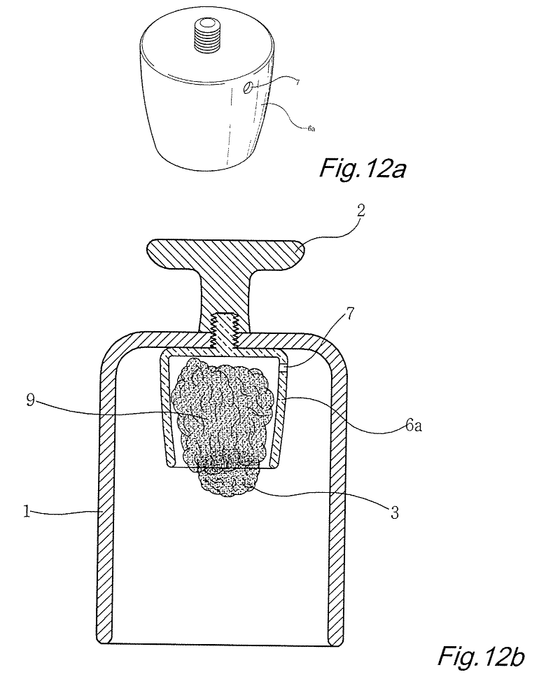

FIG. 12 shows a cupping jar with a metal lamp that is fixedly provided in the cup portion 1, not in the handle portion 2, to protrude inwardly. A groove at a lower end of the metal lamp 6a is fixed to the body of the cupping jar. The lamp is formed of a metal, and an alcohol absorbing opening 7 is provided in a body portion. In addition, in the metal lamp, an alcohol-soaked cotton 9 and a combustion core 3 are provided.

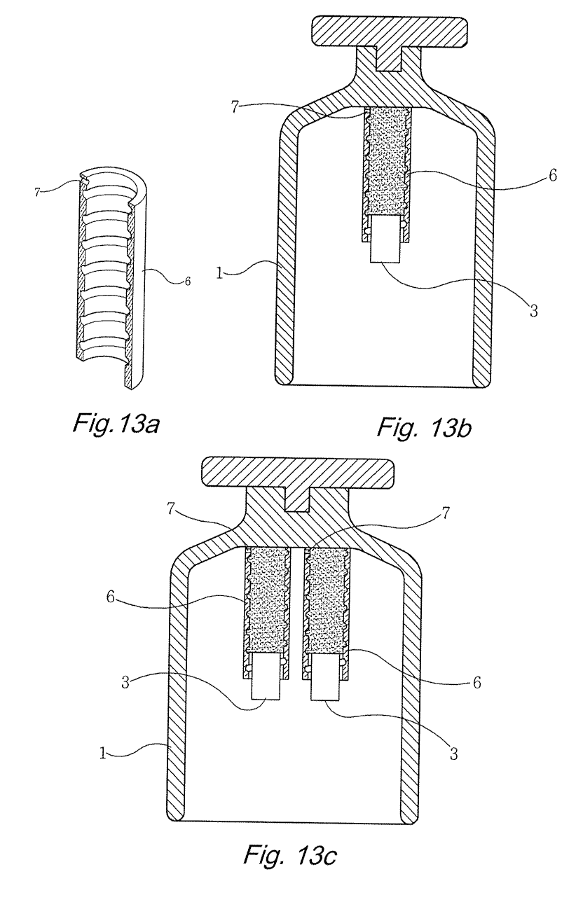

FIG. 13 is an explanatory view of a lamp in the cupping jar according to another embodiment of the invention. The lamp 6 may have a bottle-like shape, for example, similar to an alcohol lamp. However, the invention is not limited thereto. For example, the lamp 6 may have a cylindrical shape. In addition, the inner wall surface of the lamp 6 may be uneven or jagged such that the combustion core 3 and the alcohol-soaked cotton 9 are not separated from the lamp 6. In FIG. 13a, an uneven shape, such as a groove, is shown as an example for the inner wall surface. In FIG. 13c, there are two lamps and two combustion cores in one cupping jar.

1. A fire cupping jar comprising:
a cup portion 1; and
a lamp 6, 6a that is provided in the cupping jar,
wherein the lamp includes a storage unit that is filled with an absorbent for absorbing a volatile combustible material, and an ignition unit.

2. The fire cupping jar of claim 1, wherein the ignition unit includes a combustion core 3, 3a.

3. The fire cupping jar of claim 1, further comprising an ignition device.

4. The fire cupping jar of claim 1, further comprising a handle portion 2.

5. The fire cupping jar of claim 4, wherein the lamp 6 is provided in the handle portion 2.

6. The fire cupping jar of claim 3, wherein the lamp 6, 6a protrudes toward an internal space of the cup portion 1.

7. The fire cupping jar of claim 1, wherein the lamp 6a is formed of a metal, and wherein the lamp 6a is provided in an internal space of the cup portion.

8. The fire cupping jar of claim 2, wherein the number of the lamps 6, 6a is at least 2 and/or the number of the combustion cores 3, 3a is at least 2.

9. The fire cupping jar of claim 1, further comprising:
a ventilating opening 4 that is provided in the cup portion 1; and
an opening/closing unit 20 that opens/closes the ventilating opening 4.
